(19) United States
(12) Patent Application Publication
(10) Pub. No.: US 2018/0038815 A1
(43) Pub. Date: Feb. 8, 2018

(54) Nanotube-Based Biosensor for Pathogen Detection

(71) Applicant: Northeastern University, Boston, MA (US)

(72) Inventors: April GU, Newton, MA (US); Nimet YILDIRIM, Malden, MA (US); Jinyoung LEE, Seoul (KR); Hanchul CHO, Yongin, Gyeonggi-do (KR); Ahmed BUSNAINA, Needham, MA (US)

(21) Appl. No.: 15/537,080

(22) PCT Filed: Dec. 16, 2015
(86) PCT No.: PCT/US2015/066213

§ 371 Date: Jun. 16, 2017

Related U.S. Application Data
(60) Provisional application No. 62/092,534, filed on Dec. 16, 2014.

(51) Int. Cl. G01N 27/12; G01N 33/543; G01N 33/53; B01L 3/00; G01N 33/569; G01N 33/18; C12Q 1/68

(52) U.S. Cl. CPC G01N 27/12; G01N 33/18; G01N 33/5438; C12Q 1/6825; G01N 33/54353; B01L 3/502715; B01L 3/502761; G01N 33/56916; G01N 33/56983; G01N 33/5308; C12Q 1/6804; G01N 1/28

(57) ABSTRACT

A simple and highly sensitive single walled carbon nanotube (SWNT) sensor is provided for detection of a variety of analytes, including small molecules, macromolecules, and pathogens. The high sensitivity, specificity, stability, and rapid operation of the sensor render it useful for detection and quantification of low level contaminants such as pharmaceuticals and pathogens in environmental samples, including wastewater and natural bodies of water.

### CROSS REFERENCE TO RELATED APPLICATIONS

### Background

Monitoring of waste water and natural bodies of water is needed in order to protect people from toxic or dangerous chemicals and infectious diseases, such as those caused by enteric pathogens. For example, the presence and amount of Escherichia coli are good indicators for potential enteric pathogens in waters. As another example, adenovirus infection is a waterborne viral infection and an important cause of human morbidity worldwide. The traditional detection method for E coli by counting colonies on culture plates is arduous and time consuming, requiring more than 24 hours. Polymerase chain reaction (PCR), quantitative real-time PCR (qPCR), and enzyme-linked immunosorbent assay (ELISA) methods have improved both the speed and sensitivity of pathogen detection compared with detection by the traditional culturing method. However, PCR techniques have a high risk of false results owing to inhibition by components of the sample and a complicated pretreatment process, such as extraction of the pathogen DNA. The ELISA technique requires certain labeled antibodies which add cost, and the assays involve time consuming steps. Therefore, simple methods for the rapid and sensitive detection and quantification of pathogens and chemicals in water samples are urgently needed for public health protection.

### SUMMARY OF THE INVENTION

The invention provides a simple and highly sensitive single walled carbon nanotube (SWNT) sensor for detection of a variety of analytes, including small molecules, macromolecules, and pathogens. The high sensitivity, specificity, stability, and rapid operation of the sensor render it very useful for detection and quantification of low level contaminants such as pharmaceuticals and pathogens in environmental samples, including wastewater and natural bodies of water.

One aspect of the invention is a sensor for quantification of an analyte in a sample. The sensor includes: a substrate; a pair of metal electrodes deposited onto a surface of the substrate with a gap between the electrodes; and a bridge contacting both electrodes of the pair and forming a conductive pathway between the electrodes and across the gap. The bridge comprises or consists of one or more single walled carbon nanotubes (SWNT) which are non-covalently functionalized with a recognition agent capable of specifically recognizing the analyte. A conductometric circuit connected to the electrodes detects changes in resistance of the SWNT in relation to an amount of analyte present in the sample.

In embodiments of the sensor, the recognition agent is an antibody, a nucleic acid aptomer, or a nucleic acid probe that hybridizes to a nucleic acid aptomer. In embodiments, the recognition agent is covalently attached to a coupling agent that is non-covalently attached to the SWNT via π-π stacking interactions. In embodiments, the coupling agent is 1-pyrenebutanoic acid succinimidyl ester. In embodiments, the sensor is configured as a microfluidic or nanofluidic device. In embodiments, the sensor is capable of providing quantification of an analyte in less than 30 min. In embodiments, the sensor is produced by a nanoimprinting process. In embodiments, the analyte is a bacterium, and the sensor is capable of quantifying the presence of the bacterium at a concentration from 1 to about 1,000,000 CFU/mL in the sample. In embodiments, the analyte is a virus, and the sensor is capable of quantifying the virus at a concentration of 10-10,000 PFU/mL in the sample. In embodiments, the analyte is a pharmaceutical, a hormone, a toxin, or a heavy metal. In embodiments, the sample is an environmental sample or a bodily fluid from a subject. In embodiments, the sensor shares a common substrate with one or more other sensors that are capable of quantifying the same analyte or a different analyte.

Another aspect of the invention is a system for quantifying an analyte. The system includes the sensor described above and one or more additional devices to assist in quantifying the analyte. The additional devices can be, for example, a sensor reading device, a receiver, a transmitter, a display, a programmable processor, and/or a sample processing module.

Yet another aspect of the invention is a method of quantifying an analyte. The method includes the steps of: (a) providing the sensor described above, or the system described above, and a sample suspected of containing the analyte, wherein the recognition agent of the sensor is a nucleic acid probe that hybridizes to a nucleic acid aptamer that specifically binds the analyte; (b) optionally conditioning the sample by filtration, dilution, concentration, dialysis, centrifugation, or another method; (c) contacting the sample, or the conditioned sample, with the aptamer and allowing the aptamer to bind to the analyte; (d) separating unbound aptamer from the analyte; (e) hybridizing the unbound aptamer obtained in step (d) to the nucleic acid probe in the sensor; and (f) determining a change in conductance or resistance of the SWNT in the sensor, from which the concentration of analyte in the sample is determined and the analyte is thereby quantified.

In embodiments of the method, step (f) includes applying a series of different step voltages and measuring the current at each voltage. In embodiments, the method further includes calibrating the sensor using a series of standard solutions having known concentrations of the analyte. In embodiments, the method is capable of quantifying the analyte in less than 30 min. In embodiments, the analyte is a bacterium, and the method provides a linear response over the range from about 1 to about 1,000,000 CFU/mL using a plot of log(bacteria concentration) vs. ΔR/R₀, where R₀ is the SWNT resistance prior to adding the sample, and ΔR is the SWNT resistance in the presence of the sample minus R₀. In embodiments, the analyte is a virus, and the method provides a linear response over the range from about 10 to about 10,000 PFU/mL using a plot of log(virus concentration) vs. ΔR/R₀, where R₀ is the SWNT resistance prior to adding the sample, and OR is the SWNT resistance in the presence of the sample minus R₀. In embodiments, the method is capable of providing quantification of an analyte in less than 30 min. In embodiments, the analyte is a pharmaceutical, a hormone, a toxin, or a heavy metal. In embodiments, the sample is an environmental sample or a bodily fluid from a subject.

Still another aspect of the invention is a method of quantifying an analyte. The method includes the steps of: (a) providing the sensor described above, or the system described above, and a sample suspected of containing the analyte, wherein the recognition agent of the sensor is an antibody that specifically binds to the analyte; (b) optionally conditioning the sample by filtration, dilution, concentration, dialysis, centrifugation, or another method; (c) contacting the sample, or the conditioned sample, with the SWNT of the sensor and allowing the analyte to bind to the antibody; and (d) determining a change in conductance or resistance of the SWNT in the sensor, from which the concentration of analyte in the sample is determined and the analyte is thereby quantified.

In embodiments of the method, step (d) includes applying a series of different step voltages and measuring the current at each voltage. In embodiments, the method further includes calibrating the sensor using a series of standard solutions having known concentrations of the analyte. In embodiments, the method is capable of quantifying the analyte in less than 30 min. In embodiments, the analyte is a bacterium, and the method provides a linear response over the range from about 1 to about 1,000,000 CFU/mL using a plot of log(bacteria concentration) vs. ΔR/R₀, where R₀ is the SWNT resistance prior to adding the sample, and ΔR is the SWNT resistance in the presence of the sample minus R₀. In embodiments, the analyte is a virus, and the method provides a linear response over the range from about 10 to about 10,000 PFU/mL using a plot of log(virus concentration) vs. ΔR/R₀, where R₀ is the SWNT resistance prior to adding the sample, and ΔR is the SWNT resistance in the presence of the sample minus R₀. In embodiments, the method is capable of providing quantification of an analyte in less than 30 min. In embodiments, the analyte is a pharmaceutical, a hormone, a toxin, or a heavy metal. In embodiments, the sample is an environmental sample or a bodily fluid from a subject.

Another aspect of the invention is a method of fabricating the sensor described above. The method includes the steps of: (a) depositing a pair of electrodes on an insulating surface of a substrate, with a gap between the electrodes; (b) depositing one or more SWNT to form a conductive bridge between the electrodes and across the gap; (c) functionalizing the SWNT non-covalently with a recognition agent capable of specifically recognizing the analyte. A conductometric circuit connected to said electrodes detects changes in resistance of the SWNT in relation to an amount of analyte present in the sample.

In embodiments of the method, one or more SWNT are deposited in step (b) using an electric field-assisted directed assembly process. In embodiments of the method, a coupling agent is non-covalently attached to the SWNT via π-π stacking interactions, and then the recognition agent is covalently linked to the coupling agent. In embodiments, the coupling agent is 1-pyrenebutanoic acid succinimidyl ester. In embodiments, the method further includes fabricating a conductometric circuit on the substrate.

The invention can be further summarized by the following list of items:

1. A sensor for quantification of an analyte in a sample, the sensor comprising:

a substrate;

a pair of metal electrodes deposited onto a surface of the substrate with a gap between the electrodes;

a bridge contacting both electrodes of the pair and forming a conductive pathway between the electrodes and across the gap, the bridge comprising or consisting of one or more single walled carbon nanotubes (SWNT) non-covalently functionalized with a recognition agent capable of specifically recognizing said analyte;

wherein a conductometric circuit connected to said electrodes detects changes in resistance of the SWNT in relation to an amount of analyte present in the sample.
2. The sensor of item 1, wherein the bridge comprises a plurality of aligned SWNT that are assembled on the substrate by a directed assembly method and not grown in situ.
3. The sensor of item 2, wherein the assembled and aligned SWNT comprises SWNT that do not extend the full length from one of the pair of electrodes to the other.
4. The sensor of any of the preceding items, wherein the recognition agent is an antibody, a nucleic acid aptomer, or a nucleic acid probe that hybridizes to a nucleic acid aptomer.
5. The sensor of any of the preceding items, wherein the recognition agent is covalently attached to a coupling agent that is non-covalently attached to the SWNT via π-π stacking interactions.
6. The sensor of any of the preceding items, wherein the coupling agent is 1-pyrenebutanoic acid succinimidyl ester.
7. The sensor of any of the preceding items, wherein the conductometric circuit is built into the sensor.
8. The sensor of any of the preceding items, wherein the conductometric circuit is external to the sensor.
9. The sensor of item 1 or item 7, which is configured to connect to an external sensor reading device.
10. The sensor of item 7 or item 9, further comprising a wireless transmitter.
11. The sensor of any of items 7, 9, or 10, further comprising a processor.
12. The sensor of any of items 7-11, further comprising a display.
13. The sensor of any of the preceding items, configured as a microfluidic or nanofluidic device.
14. The sensor of item 13, further comprising a sample processing module.
15. The sensor of item 13 or item 14, further comprising one or more additional components selected from the group consisting of pumps, valves, filters, membranes, microdialyzers, and fluid reservoirs.
16. The sensor of any of the preceding items capable of providing quantification of an analyte in less than 30 min.
17. The sensor of any of the preceding items that is reusable or disposable.
18. The sensor of any of the preceding items that is produced by a nanoimprinting process.
19. The sensor of any of the preceding items, wherein the substrate is flexible.
20. The sensor of any of the preceding items, wherein the analyte is a microbe.
21. The sensor of item 20, wherein the microbe is a virus, bacterium, fungus, or protist.
22. The sensor of item 21, wherein the microbe is a bacterium, and the sensor is capable of quantifying the presence of the bacterium at a concentration from 1 to about 1,000,000 CFU/mL in the sample.
23. The sensor of item 22, wherein the bacterium is Escherichia coli.
24. The sensor of item 21, wherein the microbe is a virus, and the sensor is capable of quantifying the virus at a concentration of 10-10,000 PFU/mL in the sample.
25. The sensor of item 24, wherein the virus is adenovirus.
26. The sensor of any of items 1-19, wherein the analyte is a pharmaceutical, a hormone, a toxin, or a heavy metal.
27. The sensor of any of items 1-19, wherein the analyte is a macromolecule.
28. The sensor of any of items 1-19, wherein the sample is an environmental sample.
29. The sensor of any of items 1-19, wherein the sample is wastewater, tapwater, or drinking water.
30. The sensor of any of the preceding items, wherein the sample is a bodily fluid from a subject.
31. The sensor of any of the preceding items that shares a common substrate with one or more other sensors, the other sensors capable of quantifying said analyte or a different analyte.
32. A system for quantifying an analyte, the system comprising the sensor of any of the preceding items and one or more additional devices to assist in quantifying the analyte.
33. The system of item 32, comprising a sensor reading device.
34. The system of item 33, wherein the sensor and reading device are integrated into a single unit.
35. The system of item 33, wherein the reading device is a separate unit from the sensor.
36. The system of item 35, wherein the sensor attaches to or fits within the reading device for analysis.
37. The system of item 33, wherein the reading device comprises one or more modules selected from the group consisting of a receiver, a transmitter, a display, a programmable processor, and a sample processing module.
38. The system of any of items 33-37, wherein the reading device comprises or consists of a microfluidic or nanofluidic device.
39. A method of quantifying an analyte, the method comprising the steps of:

(a) providing the sensor of any of items 1-31 or the system of any of items 32-38 and a sample suspected of containing the analyte, wherein the recognition agent of the sensor is a nucleic acid probe that hybridizes to a nucleic acid aptamer that specifically binds the analyte;

(b) optionally conditioning the sample by filtration, dilution, concentration, dialysis, centrifugation, or another method;

(c) contacting the sample, or the conditioned sample, with the aptamer and allowing the aptamer to bind to the analyte;

(d) separating unbound aptamer from the analyte;

(e) hybridizing the unbound aptamer obtained in step (d) to the nucleic acid probe in the sensor; and

(f) determining a change in conductance or resistance of the SWNT in the sensor.

40. The method of item 39, wherein step (f) comprises applying a series of different step voltages and measuring the current at each voltage.
41. The method of item 40, wherein the voltages are in the range 0 to about 100 mV.
42. The method of item 39, further comprising calibrating the sensor using a series of standard solutions having known concentrations of the analyte.
43. The method of item 39 capable of quantifying the analyte in less than 30 min.
44. The method of item 39, wherein the analyte is a microbe.
45. The method of item 44, wherein the microbe is a virus, bacterium, fungus, or protist.
46. The method of item 34, wherein the analyte is a bacterium, and the method provides a linear response over the range from about 1 to about 1,000,000 CFU/mL using a plot of log(bacteria concentration) vs. ΔR/R0, where R0 is the SWNT resistance prior to adding the sample, and ΔR is the SWNT resistance in the presence of the sample minus R0.
47. The method of item 46, wherein the bacterium is Escherichia coli.
48. The method of item 44, wherein the microbe is a virus, and the method provide a linear response over the range from about 10 to about 10,000 PFU/mL using a plot of log(virus concentration) vs. ΔR/R0, where R0 is the SWNT resistance prior to adding the sample, and ΔR is the SWNT resistance in the presence of the sample minus R0.
49. The method of item 48, wherein the virus is adenovirus.
50. The method of item 39, wherein the analyte is a pharmaceutical, a hormone, a toxin, or a heavy metal.
51. The method of item 39, wherein the analyte is a macromolecule.
52. The method of item 39, wherein the sample is an environmental sample.
53. The method of item 39, wherein the sample is wastewater, tapwater, or drinking water.
54. The method of item 39, wherein the sample is a bodily fluid from a subject.
55. A method of quantifying an analyte, the method comprising the steps of:

(a) providing the sensor of any of items 1-31 or the system of any of items 32-38 and a sample suspected of containing the analyte, wherein the recognition agent of the sensor is an antibody that specifically binds to the analyte;

(b) optionally conditioning the sample by filtration, dilution, concentration, dialysis, centrifugation, or another method;

(c) contacting the sample, or the conditioned sample, with the SWNT of the sensor and allowing the analyte to bind to the antibody; and

(d) determining a change in conductance or resistance of the SWNT in the sensor.

56. The method of item 55, wherein step (d) comprises applying a series of different step voltages and measuring the current at each voltage.
57. The method of item 56, wherein the voltages are in the range 0 to about 100 mV.
58. The method of item 55, further comprising calibrating the sensor using a series of standard solutions having known concentrations of the analyte.
59. The method of item 55 capable of quantifying the analyte in less than 30 min.
60. The method of item 55, wherein the analyte is a microbe.
61. The method of item 60, wherein the microbe is a virus, bacterium, fungus, or protist.
62. The method of item 55, wherein the analyte is a bacterium, and the method provides a linear response over the range from about 1 to about 1,000,000 CFU/mL using a plot of log(bacteria concentration) vs. ΔR/R0, where R0 is the SWNT resistance prior to adding the sample, and ΔR is the SWNT resistance in the presence of the sample minus R0.
63. The method of item 62, wherein the bacterium is Escherichia coli.
64. The method of item 60, wherein the microbe is virus, and the method provide a linear response over the range from about 10 to about 10,000 PFU/mL using a plot of log(virus concentration) vs. ΔR/R0, where R0 is the SWNT resistance prior to adding the sample, and ΔR is the SWNT resistance in the presence of the sample minus R0.
65. The method of item 64, wherein the virus is adenovirus.
66. The method of item 55, wherein the analyte is a pharmaceutical, a hormone, a toxin, or a heavy metal.
67. The method of item 55, wherein the analyte is a macromolecule.
68. The method of item 55, wherein the sample is an environmental sample.
69. The method of item 55, wherein the sample is wastewater, tapwater, or drinking water.
70. The method of item 55, wherein the sample is a bodily fluid from a subject.
71. A method of fabricating the sensor for quantifying an analyte, the method comprising the steps of:

(a) depositing a pair of electrodes on an insulating surface of a substrate, with a gap between the electrodes;

(b) depositing one or more SWNT to form a conductive bridge between the electrodes and across the gap;

(c) functionalizing the SWNT non-covalently with a recognition agent capable of specifically recognizing said analyte;

wherein a conductometric circuit connected to said electrodes detects changes in resistance of the SWNT in relation to an amount of analyte present in the sample.
72. The method of item 71, wherein in step (b) one or more SWNT are deposited using an electric field-assisted directed assembly process.
73. The method of item 71, wherein the recognition agent is covalently attached to a coupling agent that is non-covalently attached to the SWNT via π-π stacking interactions.
74. The method of item 73, wherein the coupling agent is 1-pyrenebutanoic acid succinimidyl ester.
75. The method of item 71, further comprising fabricating a conductometric circuit on the substrate.

### BRIEF DESCRIPTION OF THE DRAWINGS

FIG. 1 shows a schematic representation of an embodiment of a sensor according to the present invention.

FIG. 2 depicts the interaction of 1-pyrenebutanoic acid succinimidyl ester with a single walled carbon nanotube by π-π stacking. (Chen et al., 2001)

FIG. 3A is a photograph of an array of six sensors of the invention fabricated on a common substrate. Each sensor has two gold electrodes bridged by a highly aligned bundle of SWNT, which are shown enlarged in the electron micrograph of FIG. 3B.

FIG. 4 shows a schematic illustration of a competition assay for detecting the antibiotic oxytetracycline in an aqueous sample using a DNA aptamer and a sensor of the invention functionalized with a corresponding DNA probe.

FIG. 5 shows the response (change in resistance) of an oxytetracycline-specific aptamer sensor as a function of oxytetracycline concentration in the sample. The inset shows the linear portion of the oxytetracycline standard curve.

FIG. 6 shows the specificity of an oxytetracycline-specific aptamer sensor for oxytetracycline over other antibiotics.

FIG. 7A shows the repeatability of oxytetracycline standard curves after several cycles of sensor regeneration, and FIG. 7B shows the effect of aging of the sensor for up to 30 days on the oxytetracycline standard curve.

FIG. 8 shows a schematic illustration of a direct binding assay for detecting adenovirus using a sensor functionalized with an adenovirus-specific antibody.

FIG. 9 shows the linear portion of a standard curve for adenovirus detection using the assay of FIG. 8.

FIG. 10 shows the specificity of the assay of FIG. 8 for adenovirus over other viruses and bacteria.

FIG. 11 shows a schematic illustration of a competition assay for detecting E. coli cells in an aqueous sample using a DNA aptamer and a sensor of the invention functionalized with a corresponding DNA probe.

FIG. 12 shows the linear portion of a standard curve for E. coli O157 H:7 detection using the assay of FIG. 11.

FIG. 13 shows the specificity of the E. coli O157 H:7-specific aptamer sensor with respect to other E. coli strains and other bacterial species.

FIG. 14 shows the stability of the E. coli O157 H:7-specific aptamer sensor as a function of time.

### DETAILED DESCRIPTION OF THE INVENTION

The invention provides a simple and highly sensitive single walled carbon nanotube (SWNT) based sensor for a wide variety of analytes, including chemicals of low molecular weight (e.g., <1500 Da), macromolecules, and microbes, including pathogenic microbes. The sensor relies on functionalization of the SWNT with analyte-specific aptamers or antibodies. The sensor can be used with different assay formats, including a direct detection mode, where the analyte binds to a recognition agent (e.g., an antibody or aptamer) attached to the SWNT, or it can be used in an indirect competitive detection mode, in which a sample is mixed with an aptamer that specifically binds to the analyte, and unbound aptamer is detected by its ability to hybridize to a complementary or partially complementary probe sequence, which is attached to the SWNT and serves as recognition agent. The assays produce linear standard curves over a wide range of concentrations, e.g., down to the low nM level for small molecules and down to about 1 CFU/mL for bacteria and 1 PFU/mL for viruses. The detection system can be regenerated successfully with low concentrations of SDS or NaOH solutions over 100 times without significant deterioration of performance. Specificity is high, generally more than 80% over related analytes such as other viruses and bacteria. The sensor also allows rapid determinations of analyte concentration in a sample, generally in less than 1 hour and often in less than 30 minutes.

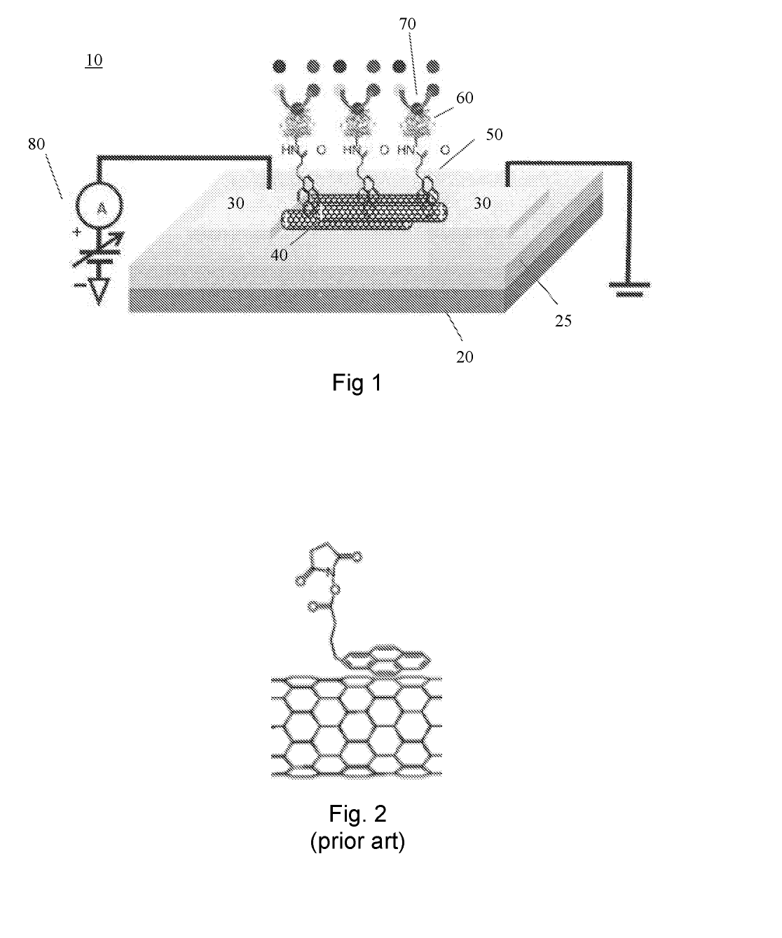

FIG. 1 shows a schematic of an embodiment of a sensor of the invention. Sensor device 10 includes substrate 20, with an optional coating of insulating layer 25. Deposited on the substrate, or insulating layer if present, is pair of conductive electrodes 30, separated by a gap which is bridged by bundle of SWNT 40 connecting the two electrodes. The SWNT are functionalized through non-covalently attached coupling agent 50, to which is covalently bound recognition agent 60, which is specific for selected analyte 70. The electrodes are electrically coupled to detection circuit 80, which is preferably a conductometric circuit, i.e., a circuit that is suitable for measuring conductance, and changes therein, of the SWNT bridge. The circuit also measures resistance, and changes therein, which are the inverse of conductance.

The sensor can detect the presence or absence of, and quantify, within certain limits of detection, any analyte for which a specific recognition element can be obtained, wherein the recognition element can be coupled to SWNT resulting in an increase in resistance (or decrease in conductance) of the SWNT in the presence of the analyte. Examples of suitable analytes are chemicals (i.e., small organic molecules and certain inorganic compounds or elements, including any type of small molecule drug, toxic substances, food components, pesticides, insecticides, and the like), macromolecules (peptides, polypeptides, proteins, glycoproteins, nucleotides, nucleic acids, carbohydrates, polysaccharides, and the like), and cells (cells of a human or animal body, microbes such as viruses, bacteria, fungi, and protists, including pathogenic or diseased varieties thereof).

The analyte is present in a sample, which is preferably a liquid sample, although contents of solid or gaseous samples can be transferred into liquid solutions or suspensions for analysis. The liquid sample can be, for example, an environmental sample, such as from a natural body of water, or collected rain, snow, or ice (which can be melted to provide liquid), or it can be a waste liquid or effluent from an industrial plant or a municipal waste treatment system, or it can be purified or treated water from a potable water supply system, or drinking water in bottled or other form. The liquid sample can also be any type of bodily fluid or secretion from a human or animal body, such as blood, serum, plasma, or urine. If the concentration or form of the liquid sample is not suitable for direct assay by the sensor, the sample can be filtered, diluted, concentrated, dialyzed, precipitated, freeze-dried and reconstituted, or otherwise conditioned prior to analysis.

In order for the SWNT to be suitably functionalized, a coupling agent is non-covalently bound to the SWNT. Preferred coupling agents interact with SWNT by π-π interactions, which form a tight but non-covalent bond to the outer wall of the SWNT. One example of a suitable coupling agent is 1-pyrenebutanoic acid succinimidyl ester (PBSE), and related analogues or derivatives. PBSE is a versatile coupling agent which attaches to SWNT through non-covalent π-π stacking that does not damage the geometric and electronic configuration of the SWNT. Its aromatic hydrophobic domain spontaneously binds to the hydrophobic SWNT sidewalls through non-covalent molecular adsorption. Furthermore, the π electrons enhance the electronic and thermal properties of SWNT. In addition, the hydrophilic domain of PBSE, the succinimidyl ester group, provides a reactive amine site that can provide covalent attachment sites for attaching a variety of biological and non-biological ligands to the PBSE and thus to the SWNT. Thus, any analogue or derivative of PBSE that preserves the π-π stacking interaction, usually through an unsaturated 6-membered carbon ring or similar aromatic ring structure, as well as a group subject to nucleophilic attack by amino groups or other groups on the recognition agent (e.g., an aptamer or antibody molecule) can be used. Preferably, the coupling agent also has a linker portion that separates the aromatic portion from the leaving group, in order to provide flexibility and reactivity with the recognition group. For example, the C4 linker of PBSE can be shortened to a C2 or C3 linker or lengthened up to a C12 linker; preferably, it is a C3, C4, or C5 linker. FIG. 2 shows the molecular interaction between PBSE and an SWNT, which leads to π-π stacking. It is understood that a plurality of coupling agent moieties will attach along the length of each SWNT, so as to provide a sufficient density of functionalization. For example, each nanotube can have 10 or more, 100 or more, 1000 or more, 10000 or more, 100000 or more, 1 million or more, 10 million or more, 100 million or more coupling agent molecules attached via π-π interactions along its length.

While the recognition agent can be any binding molecule or ligand that forms a stable non-covalent or covalent bond with the analyte, or a molecular component of the analyte, preferred recognition agents are aptamers of DNA or other nucleic acids capable of hybridizing and forming double stranded molecules, and antibodies. Suitable antibodies include intact natural antibodies and analyte-binding fragments thereof, such as single chain antibodies, nanobodies, diabodies, F_{ab} fragments, recombinant antibodies, and the like. Methods are known in the art for routinely generating both aptamers and antibodies with a high degree of specificity for binding practically any analyte. It is understood that binding of the recognition agent to the analyte can be routinely optimized with regard to time, concentration of analyte and recognition agent, and solution conditions.

The detection range and limit of detection (LOD) of the sensor for a given analyte will depend on the design of the assay as well as the quality of the recognition agent and chemistry of the coupling agent. In general, a broad range of linear dependence on analyte concentration can be obtained for chemicals in the range from about 1 nM to about 1 mM, or from about 1 nM to about 1 μM, or from about 1 μM to about 1 mM, or from about 10 nm to about 1 μM, or from about 100 nM to about 100 μM can be achieved. For bacteria, a linear detection range can be obtained over about 1 CFU/mL to about 1 million CFU/mL, or about 10 CFU/mL to about 100,000 CFU/mL, or about 10 CFU/mL to about 1 million CFU/mL, or less than about 100,000 CFU/mL, less than about 10,000 CFU/mL, less than about 1,000 CFU/mL, or less than about 100 CFU/mL. For viruses, a linear detection range can be obtained over about 1 PFU/mL to about 1 million PFU/mL, or about 10 CPU/mL to about 100,000 PFU/mL, or about 10 PFU/mL to about 1 million PFU/mL, or less than about 100,000 PFU/mL, less than about 10,000 PFU/mL, less than about 1,000 PFU/mL, or less than about 100 PFU/mL. The LOD for bacteria can be about 1, 2, 5, 10, or 20 CFU/mL, and for viruses can be about 1, 2, 5, 10, or 20 PFU/mL.

Detection assays according to the invention can be carried out in a short period of time, such as less than one hour, less than 50 min, less than 40 min, less than 30 min, less than 20 min, or less than 10 min.

### EXAMPLES

### Example 1

### Fabrication of an SWNT-Based Sensor by a Nanoimprinting Process

A flexible biosensor was fabricated by directed assembly and printing transfer using a reusable damascene template. The method was similar to that described in Cho et al., 2015. The damascene template was fabricated as described in WO2013/070931. The damascene template and a plain gold template were used as electrode and counter electrode, respectively. Both the damascene template and counter electrode were immersed into a suspension of SWNT (0.001 wt % semiconducting SWNT). A DC power supply was used to apply a potential of 2 to 2.5V between the two electrodes, with a positive potential at the damascene template. Negatively charged SWNT were attracted onto the positively-charged conductive patterns on the damascene template. The template was then withdrawn at a constant pulling speed of 5 mm/min to 10 mm/min using a dip coater, while keeping the voltage on. Highly dense and uniform SWNT assembly was achieved on the conductive patterns in the damascene template.

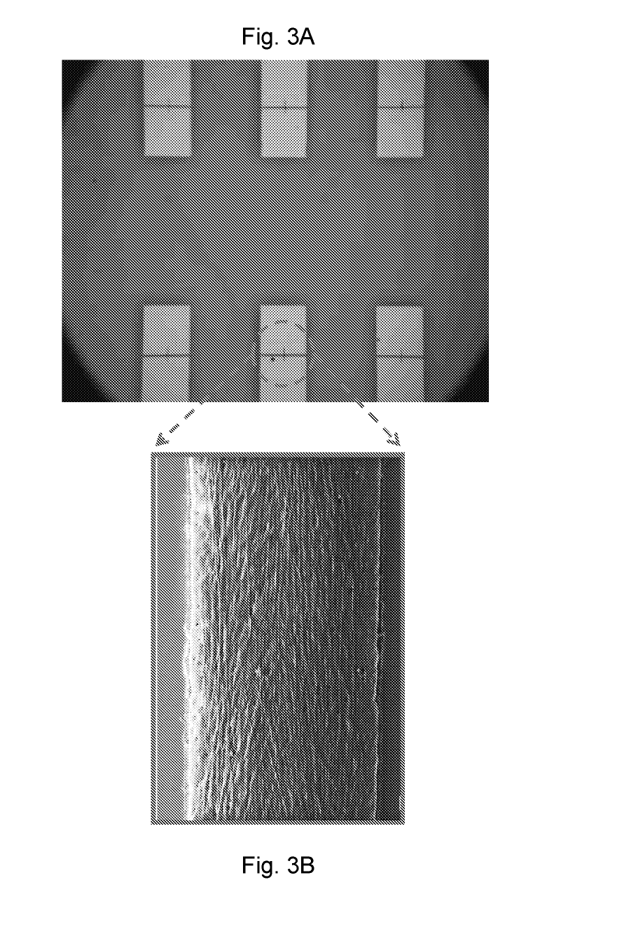

Assembled SWNT were then transferred onto a polyethylene-naphthalate (PEN) film (Teonex Q65A, Teijin DuPont) using the nanoimprinting technique. To improve the surface energy of the PEN film so as to increase the transfer yield, the PEN film was pretreated with an oxygen inductively coupled plasma (ICP). A nanoimprint tool was utilized for the printing transfer process. So as to be above the glass transition temperature of PEN (115° C.), a process temperature of 160° C. was used, and 170 psi pressure was applied to the template and PEN film for 1 min. After cooling to room temperature, the film was gently peeled off from the template. Above the glass transition temperature, the PEN film engulfed the assembled SWNT tightly, and high yield transfer was achieved. Metal electrodes were fabricated on the PEN-based sensor as layers of Cr and Au (5 nm and 100 nm, respectively) which covered and contacted the SWNT bundles deposited by nanoimprinting. The electrodes were fabricated using photolithography, electron beam deposition, and a lift off process. An array of completed sensors is shown in FIG. 3A, and an enlarged view of the SWNT bridge is shown in FIG. 3B.

### Example 2

### Quantification of Oxytetracycline (OTC) Using an SWNT-Based Sensor

An indirect competitive mode sensing mechanism was used, which included steps of pre-mixing, measurement of resistance change, and regeneration. The indirect detection mode was deemed to be the best in view of the potential problems caused by the large number of contaminants in waste water samples and to a high non-specific adsorption onto sensor surface. Additionally, using an indirect detection mode with non-immobilized aptamers provides much more relaxed binding between OTC and the aptamers, and also reduces the required binding time. The sensor's sensing time, sensitivity, specificity, resistance to background interference and reusability were evaluated. The developed OTC sensing system exhibited a sensitive response concentration range and detection limit comparable to OTC levels in environmental water and therefore can be used for on-site analysis without any pre-concentration or treatment steps.

OTC was purchased from Sigma-Aldrich (MO, USA), and the linker; 1-pyrenebutanoic acid-succinimidyl ester (PBSE) was purchased from Invitrogen (CA, USA). A single-stranded DNA aptamer with binding specificity for OTC was isolated by a SELEX process from a random ssDNA library (Javed H. Niazi, Lee, Kim, & Gu, 2008) and, together with the corresponding probe-DNA, was purchased from Integrated DNA Technologies (USA). The sequences for the aptamer and the aminated probe-DNA were: 5′-GGAATTCGCTAGCACGTTGACGCTGGTGCCCGGTTGTGGTGCGAGTGTTGTGTGGATC CGAGCTCCACGTG-3 (aptamer, SEQ ID NO:1) and 5′-/5AmMC6/CACGTGGAGCTCGG ATCCACACAACA-3′ (probe, SEQ ID NO:2). Both aptamer and probe DNA were dissolved in 100 mM PBS and kept frozen at −20° C. for storage. A buffer solution of 100 mM PBS was used for dissolving all DNA sequences, OTC, and water sample effluents, which contained 200 mM NaCl, 25 mM KCl, 10 mM MgCl₂ and had a pH of 7.4. For sensor specificity tests, the antibiotics amoxicillin, diaminofen, genomiycin, amphotericin, and ciprofloxacin (Thermo Fisher Scientific Inc. PA, USA) were tested. For sidewall functionalization of CNT with PBSE, the transfer-printed SWNT electrodes were soaked in a PBSE solution (2 mg/ml PBSE in N,N dimethylformamide) for 2 h at room temperature, washed thoroughly with N,N-DMF to remove excess PBSE, and then with deionized water. The IV profile of the linker-modified SWNT was observed. Probe-DNA was dissolved in bicarbonate buffer (0.1 mM, pH 9.2) and then stored at −20° C. until use. For probe-DNA immobilization, PBSE-modified SWNT electrodes were incubated with 0.01 and 0.05 mg/ml probe DNA for overnight at 4° C. Excess probe-DNA was then removed by washing with phosphate buffer and deionized water, and the IV profile of the electrode was tested immediately.

Sensor resistance measurements were conducted using a probe station (4156C, Agilent Technologies Co., Ltd., USA) at ambient conditions. The electrical properties of the probe-modified SWNT device upon introduction of OTC aptamer was measured using meter probes (SE-TL, SIGNATONE, USA) connecting with source and drain (the gold electrodes). A pulsed source-drain bias of 0 to 100 mV was maintained throughout the measurements of sensor resistance, with a pulse width of 1.0 s. The plates were cleaned thoroughly with PBS (pH 7.4) and deionized water, and then dried with nitrogen gas after the electrical measurements for each sample.

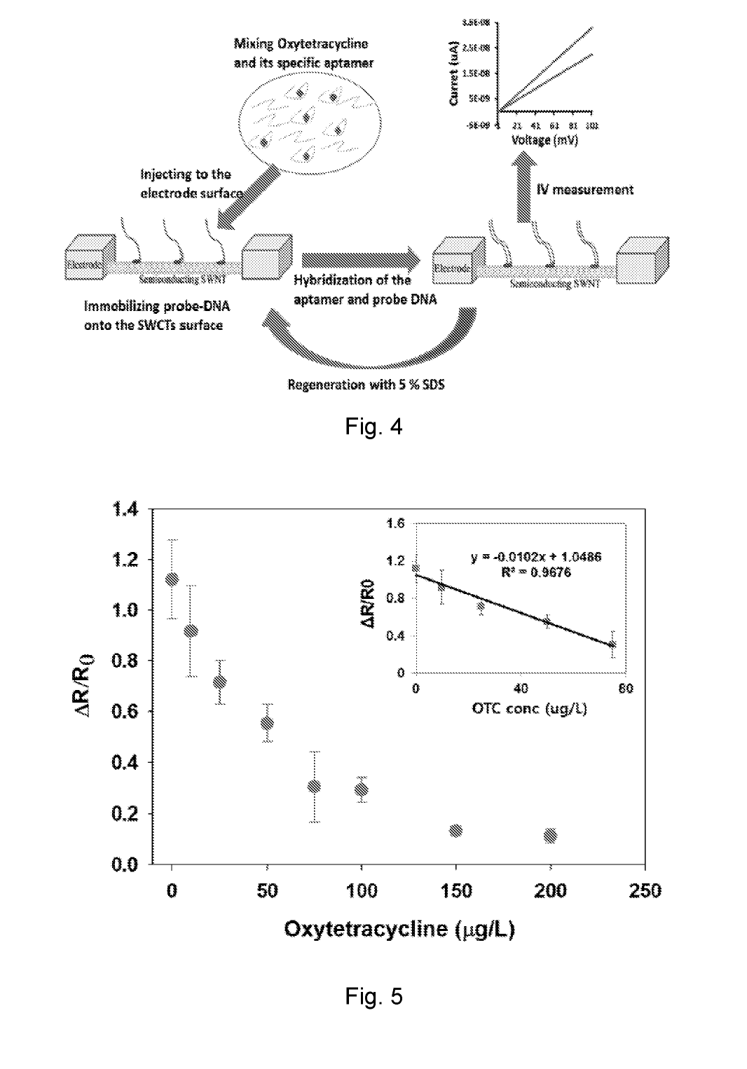

The assay using the SWNT aptamer-based sensor for detection of OTC is represented in FIG. 4. The indirect competitive detection mode included a pre-mixing step to incubate samples containing various concentrations of OTC with a fixed amount of OTC-aptamer. Upon the completion of binding between OTC and its specific aptamer, the remaining free aptamer concentration was inversely proportional to that of OTC in the water sample. The sample mixture was then injected onto the gold chip surface; the remaining free aptamers were allowed to bind to the immobilized probe-DNA which was complementary to a certain section of the OTC-aptamer (reaction time of 3 min). The IV relation was recorded before and after OTC+aptamer mixture injection onto the sensor surface, and resistance (R) differences were observed for each experiment. ΔR/R₀ values were calculated for each experiment; ΔR=R after injection minus R before injection. R₀=R before injection.

Different concentrations of OTC (0, 10, 25, 50, 75, 100, 150, and 200 μg/L) and 100 μg/L OTC-aptamer were mixed for 6 minutes and injected onto the gold chip surface. Before this injection the IV profile was observed for the gold electrode. After 3 minutes to allow for hybridization of the free aptamers to the probe DNA (immobilized on the SWNT), the IV profile of the SWNT was observed again. The normalized changes in resistance (ΔR/R₀) were calculated for each OTC concentration. The increase in the OTC concentrations in the sample and known aptamer mixture led to proportional decrease in residual free aptamer, therefore the ΔR/R₀ decrease. FIG. 5 shows the calibration curve for OTC. The error bars correspond to the standard deviations of the data points in five independent experiments, with the coefficient of variation of all the data points being within 3-21%.

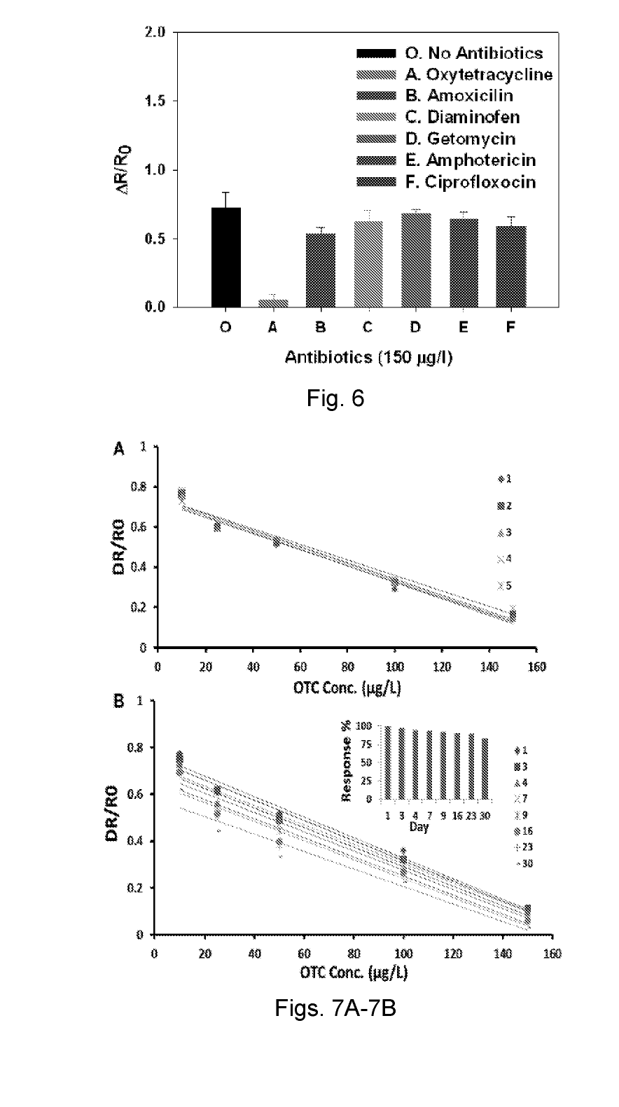

The linear range of OTC detection was between 10 and 75 μg/L (20-325 nM), and the lower detection limit (LOD) was determined to be 1.125 μg/L (2.5 nM), based on the dose response curve that is 3 times the signal standard deviation. Sensor specificity was assessed via comparison of the sensor signals of OTC with those other antibiotics, all at 150 μg/L, and each data value the average of three independent experimental results. According to the results shown in FIG. 6, with competitive detection mode sensing mechanism, the other antibiotics produced about 10% to 20% decrease in ΔR/R₀ values compared to control (no antibiotics), compared to about 95% decrease for OTC. The effects of other antibiotics are assumed to result from non-specific adsorption onto the SWNT surface.

The repeatability and stability of the sensor for OTC detection were investigated, and the results are shown in FIGS. 7A (repeatability) and 7B (stability). For assessment of reusability, the ΔR/R₀ responses for five different OTC concentrations were determined, with the sensing surface regenerated with a 0.5% SDS solution for 5 min and washed with a PBS solution (pH 7.2) between determinations. Less than 20% signal reduction was observed after five determinations. For the stability assessment, the ΔR/R₀ responses for five different OTC concentrations were determined as three daily measurements over a 30-day period. The response decreased less than 10% over 30 days.

### Example 3

### Quantification of Adenovirus Using an SWNT-Based Sensor

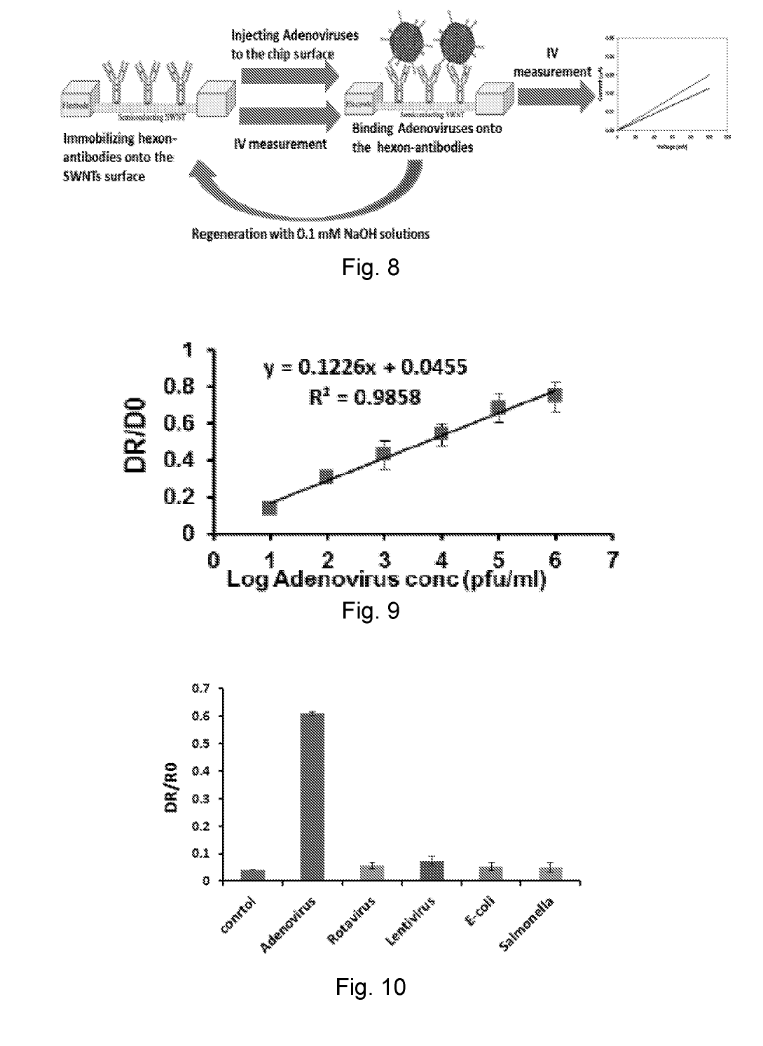

The sensing mechanism of the antibody-based SWNT biosensor for direct detection of adenovirus is represented in FIG. 8. The sensing mechanism first begins with an IV measurement before the adenovirus injection onto the SWNT. Next, different amounts of adenovirus solutions were injected onto the SWNT surface and allowed to bind the surface immobilized hexon antibodies. After the binding was completed, the final IV measurement was performed, and the resistance differences were calculated. To reuse the sensor, the sensing surface was regenerated with a 0.1 mM NaOH solution for 2 min and then washed with a PBS solution (pH 7.2). Other aspects of the sensor and measurements were as described in Example 2.

Adenovirus hexon mouse anti-virus monoclonal (3G0) antibody-LS-055826 was purchased from LifeSpan Biosciences, Inc. Seattle, Wash. Adenovirus serotypes 5 (rAd5), Rotavirus Wa, and Salmonella Typhimurium (CGMCC 1.1589) were purchased from SinoGenoMax Co., Ltd. (Beijing, China). Lentivirus (LV-CMV-vector control) was purchased from KeraFAST, Inc. (Boston, Mass.). E-coli 0157:H7 strain was kindly provided by Dr. Kim Lewis from Biology Department at Northeastern University (MA, U.S.). Human lung carcinoma cell line A549 was obtained from Prof. Rebecca Carrier's laboratory in Chemical Engineering Department at Northeastern University. Human lung carcinoma cell line A549 was cultures in the condition described by Jiang et al., 2009. A549 cells were grown in Ham's F12 medium containing 5% FBS, 2 mML-glutamine, 100U/m1 penicillin, and 100 mg/ml streptomycin. Cells were sub-cultured at 4- to 5-day intervals with a trypsin-EDTA solution. Adenovirus plaque assays using A549 cells was as described previously (Jiang et al., 2009).

A dose-response curve for adenovirus detection was determined for an adenovirus concentration from 1 PFU/mL to 10⁶ PFU/ml). FIG. 9 shows the linear range of the curve using a 10 minute binding time. Each data value is the average of five independent experimental results. The lower limit of detection was about 2 PFU/mL, based on a three times the standard deviation rule. FIG. 10 shows the results of a sensor specificity assessment. The ΔR/R₀ values for adenovirus were compared with those of other pathogen strains. Virus strains were applied at 2000 PFU/mL and bacterial strains were applied at 2000 CFU/mL. Each data value is the average of three independent experimental results. The signals for the other pathogens showed ΔR/R₀ values of about 0.05 to 0.1 compared to 0.6 for adenovirus. The signals for the other pathogens were assumed to result from non-specific binding to the SWNT.

### Example 4

### Quantification of E. coli Using an SWNT-Based Sensor

Different E. coli strains (E. coli O157 H:7, E. coli MG1655, E. coli MV1978, E. coli MV1973) were kindly provided from Professor Kim Lewis at the Antimicrobial Discovery Center in Northeastern. Bacillus cereus and Comamonas testosterone were isolated from the aeration basin of Clemson Municipal Wastewater Treatment Plant by Prof Ferdi L Hellweger's group from Civil and Environmental Engineering at Northeastern University. Recombinant Adenovirus serotype 5 (rAd5), Rotavirus Wa, and Salmonella Typhimuriu (CGMCC 1.1589) were obtained from Tsinghua University (Beijing, China). All bacteria strains were inoculated into lysogeny broth (LB) and grown for 16 h at 37° C. The cultures containing bacteria were centrifuged at 3,000 rpm for 5 min and washed with phosphate-buffered solution (PBS) (10 mM, pH 7.4) three times. The pellets were then dispersed in PBS. Serial dilutions of cultures were made in PBS; 50 μl diluted suspension were inoculated onto agar plates for enumeration, and after growing them in the same conditions, they were counted by light microscope. The bacterial densities were determined also by measuring the OD.

A DNA aptamer against E-coli O157:H7 (isolated by a SELEX process from a random ssDNA library), probe DNA, and non-specific DNA were purchased from Integrated DNA Technologies (IA, USA). The sequences were: 5′-GTC TGC GAG CGG GGC GCG GGC CCG GCG GGG GATGCGC-3 (aptamer, SEQ ID NO:3), 5′-NH₂—(CH₂)₆-GCGCATCCCCCGCCGGGCC-3′ (probe, SEQ ID NO:4), 5′-Cy5.5-CCGGTGGG TGGTCAGGTGGGATAGCGTTCCGCGTATGGCCCAGCCATCACGGGTTCGCACCA-3′ (non-specific DNA sequence used for control, SEQ ID NO:5). Aptamer, probe, and non-specific DNA oligonucleotides were dissolved in 100 mM PBS (pH 7.4) and kept frozen at −20° C. for storage.

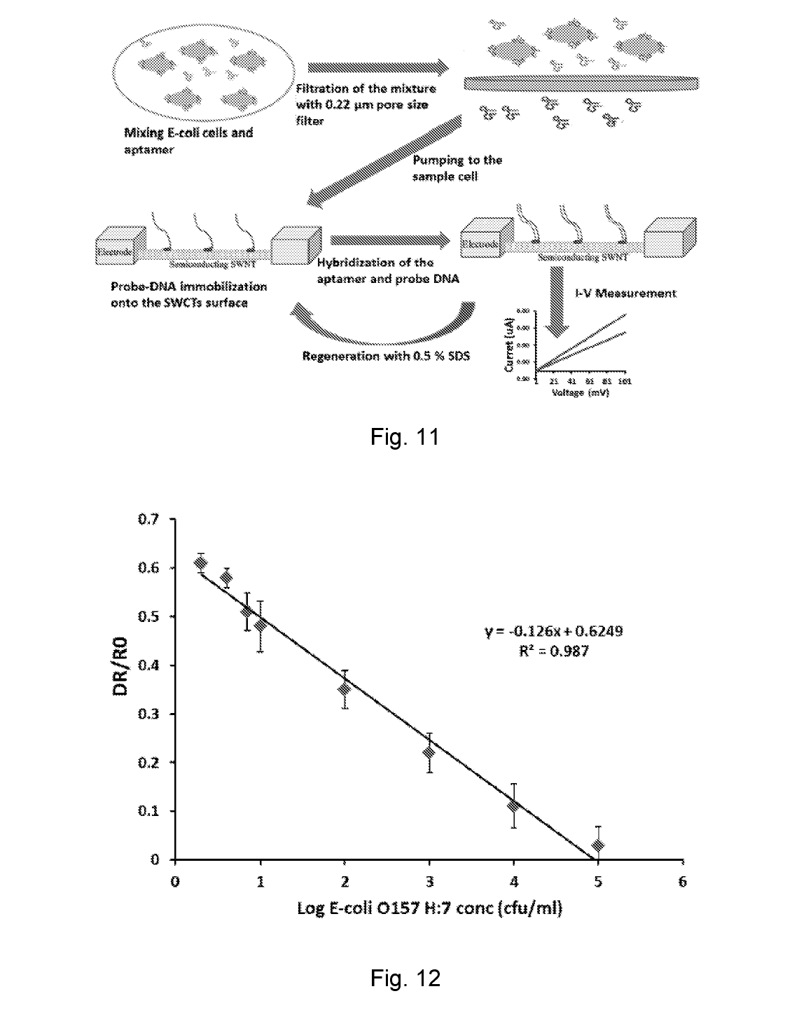

The sensing mechanism of the aptamer-based biosensor for detection of E-coli is represented in FIG. 11. An indirect detection mode was used, which included a pre-mixing step to incubate samples containing various concentrations of E-coli cells with a fixed amount of E-coli-aptamer. After a fixed time of 30 minutes, the mixture was filtered through a 0.22 μm pore filter to remove any E-coli bound aptamers. The remaining free aptamer concentration was inversely proportional to that of E-coli concentration in the water sample. After the filtration, the sample mixture was injected onto the gold chip surface, and the remaining free aptamers were allowed to bind to the immobilized probe-DNA that was complementary to a certain section of the E-coli-aptamer (reaction time was 3 min). The IV signal was recorded before and after the injection onto the sensor surface, and resistance differences values were observed for each experiment. To reuse the sensor, the sensing surface was regenerated with a 0.5% SDS solution for 5 min and washed with a PBS solution (pH 7.2). Other aspects of the sensor and measurements were as described in Example 2.

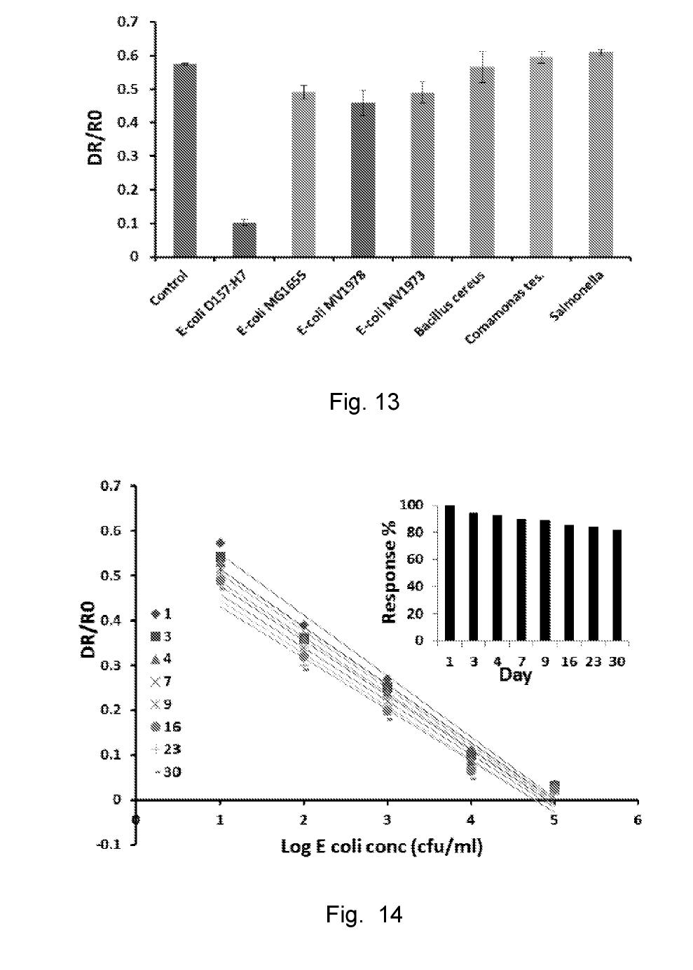

An increase in E-coli concentrations in the sample led to a proportional decrease in residual free aptamer, and therefore a proportional decrease in the resistance change. FIG. 12 shows the calibration curve for E-coli, which was normalized by expressing the signal of each standard point as the ratio to that of the blank sample containing no E-coli cells. The error bars in the figure correspond to the standard deviations of the data points in five independent experiments, with the coefficient of variation of all the data points being within 7-22%. The linear range was from 2 to 10⁵ CFU/mL, and the detection limit was 2 CFU/mL. The results of the specificity experiment are shown in FIG. 12. The results showed that the sensor had a high specificity towards the pathogenic E-coli O157:H7 strain. The control experiments used only 5 μg/mL aptamer without any pathogen strain. The other pathogen strains showed nearly 15% signal decrease, which was assumed to result from non-specific adsorption onto the SWNT surface. reusability of the DNA probe covalently immobilized to the sensing surface was evaluated over a large number (>100 assay over 30 days during this study) of assays. The stability of the sensor was evaluated by performing daily measurements over 30 days. Less than 20% signal decrease was observed in the E-colidetection procedure over the 30 day period (FIG. 13). This slight drop in resistance signal did not affect the specific response of DNA biosensor.

This application claims the priority of U.S. Provisional Application No. 62/092,534, filed 16 Dec. 2014 and entitled “Pathogen Detection in Environmental Waste Water”, the whole of which is hereby incorporated by reference.

As used herein, “consisting essentially of” allows the inclusion of materials or steps that do not materially affect the basic and novel characteristics of the claim. Any recitation herein of the term “comprising”, particularly in a description of components of a composition or in a description of elements of a device, can be exchanged with “consisting essentially of” or “consisting of”.

While the present invention has been described in conjunction with certain preferred embodiments, one of ordinary skill, after reading the foregoing specification, will be able to effect various changes, substitutions of equivalents, and other alterations to the compositions and methods set forth herein.

### REFERENCES

Chen, R. J., Zhang, Y., Wang, D., & Dai, H. (2001). Noncovalent sidewall functionalization of single-walled carbon nanotubes for protein immobilization. Journal of the American Chemical Society, 123(16), 3838-3839.
Cho, H., Somu, S., Lee, J. Y., Jeong, H., & Busnaina, A. (2015). High-rate nanoscale offset printing process using directed assembly and transfer of nanomaterials. Adv Mater, 27(10), 1759-1766. doi: 10.1002/adma.201404769.
Jiang, H., Patel, P. H., Kohlmaier, A., Grenley, M. O., McEwen, D. G., & Edgar, B. A. (2009). Cytokine/Jak/Stat signaling mediates regeneration and homeostasis in the Drosophila midgut. Cell, 137(7), 1343-1355.

1. A sensor for quantification of an analyte in a sample, the sensor comprising:
a substrate;
a pair of metal electrodes deposited onto a surface of the substrate with a gap between the electrodes;
a bridge contacting both electrodes of the pair and forming a conductive pathway between the electrodes and across the gap, the bridge comprising or consisting of one or more single walled carbon nanotubes (SWNT) non-covalently functionalized with a recognition agent capable of specifically recognizing said analyte;
wherein a conductometric circuit connected to said electrodes detects changes in resistance of the SWNT in relation to an amount of analyte present in the sample.

2. The sensor of claim 1, wherein the bridge comprises a plurality of aligned SWNT that are assembled on the substrate by a directed assembly method and not grown in situ.

3. The sensor of claim 2, wherein the assembled and aligned SWNT comprises SWNT that do not extend the full length from one of the pair of electrodes to the other.

4. The sensor of claim 1, wherein the recognition agent is an antibody, a nucleic acid aptomer, or a nucleic acid probe that hybridizes to a nucleic acid aptomer.

5. The sensor of claim 1, wherein the recognition agent is covalently attached to a coupling agent that is non-covalently attached to the SWNT via π-π stacking interactions.

6. The sensor of claim 1, wherein the coupling agent is 1-pyrenebutanoic acid succinimidyl ester.

7. The sensor of claim 1, wherein the conductometric circuit is built into the sensor.

8. The sensor of claim 1, wherein the conductometric circuit is external to the sensor.

9. The sensor of claim 1 or claim 7, which is configured to connect to an external sensor reading device.

10. The sensor of claim 7, further comprising a wireless transmitter.

11. The sensor of claim 7, further comprising a processor.

12. The sensor of claim 7, further comprising a display.

13. The sensor of claim 7, configured as a microfluidic or nanofluidic device.

14. The sensor of claim 13, further comprising a sample processing module.

15. The sensor of claim 13 or claim 14, further comprising one or more additional components selected from the group consisting of pumps, valves, filters, membranes, microdialyzers, and fluid reservoirs.

16. The sensor of claim 1 capable of providing quantification of an analyte in less than 30 min.

17. The sensor of claim 1 that is reusable or disposable.

18. The sensor of claim 1 that is produced by a nanoimprinting process.

19. The sensor of claim 1, wherein the substrate is flexible.

20. The sensor of claim 1, wherein the analyte is a microbe.

21. The sensor of claim 20, wherein the microbe is a virus, bacterium, fungus, or protist.

22. The sensor of claim 21, wherein the microbe is a bacterium, and the sensor is capable of quantifying the presence of the bacterium at a concentration from 1 to about 1,000,000 CFU/mL in the sample.

23. The sensor of claim 22, wherein the bacterium is Escherichia coli.

24. The sensor of claim 21, wherein the microbe is a virus, and the sensor is capable of quantifying the virus at a concentration of 10-10,000 PFU/mL in the sample.

25. The sensor of claim 24, wherein the virus is adenovirus.

26. The sensor of claim 1, wherein the analyte is a pharmaceutical, a hormone, a toxin, or a heavy metal.

27. The sensor of claim 1, wherein the analyte is a macromolecule.

28. The sensor of claim 1, wherein the sample is an environmental sample.

29. The sensor of claim 1, wherein the sample is wastewater, tapwater, or drinking water.

30. The sensor of claim 1, wherein the sample is a bodily fluid from a subject.

31. The sensor of claim 1 that shares a common substrate with one or more other sensors, the other sensors capable of quantifying said analyte or a different analyte.

32. A system for quantifying an analyte, the system comprising the sensor of claim 1 and one or more additional devices to assist in quantifying the analyte.

33. The system of claim 32, comprising a sensor reading device.

34. The system of claim 33, wherein the sensor and reading device are integrated into a single unit.

35. The system of claim 33, wherein the reading device is a separate unit from the sensor.

36. The system of claim 35, wherein the sensor attaches to or fits within the reading device for analysis.

37. The system of claim 33, wherein the reading device comprises one or more modules selected from the group consisting of a receiver, a transmitter, a display, a programmable processor, and a sample processing module.

38. The system of claim 33, wherein the reading device comprises or consists of a microfluidic or nanofluidic device.

39. A method of quantifying an analyte, the method comprising the steps of:
(a) providing the sensor of any of claims 1-31 or the system of any of claims 32-38 and a sample suspected of containing the analyte, wherein the recognition agent of the sensor is a nucleic acid probe that hybridizes to a nucleic acid aptamer that specifically binds the analyte;
(b) optionally conditioning the sample by filtration, dilution, concentration, dialysis, centrifugation, or another method;
(c) contacting the sample, or the conditioned sample, with the aptamer and allowing the aptamer to bind to the analyte;
(d) separating unbound aptamer from the analyte;
(e) hybridizing the unbound aptamer obtained in step (d) to the nucleic acid probe in the sensor; and
(f) determining a change in conductance or resistance of the SWNT in the sensor.

40. The method of claim 39, wherein step (f) comprises applying a series of different step voltages and measuring the current at each voltage.

41. The method of claim 40, wherein the voltages are in the range 0 to about 100 mV.

42. The method of claim 39, further comprising calibrating the sensor using a series of standard solutions having known concentrations of the analyte.

43. The method of claim 39 capable of quantifying the analyte in less than 30 min.

44. The method of claim 39, wherein the analyte is a microbe.

45. The method of claim 44, wherein the microbe is a virus, bacterium, fungus, or protist.

46. The method of claim 34, wherein the analyte is a bacterium, and the method provides a linear response over the range from about 1 to about 1,000,000 CFU/mL using a plot of log(bacteria concentration) vs. ΔR/R0, where R0 is the SWNT resistance prior to adding the sample, and ΔR is the SWNT resistance in the presence of the sample minus R0.

47. The method of claim 46, wherein the bacterium is Escherichia coli.

48. The method of claim 44, wherein the microbe is a virus, and the method provide a linear response over the range from about 10 to about 10,000 PFU/mL using a plot of log(virus concentration) vs. ΔR/R0, where R0 is the SWNT resistance prior to adding the sample, and ΔR is the SWNT resistance in the presence of the sample minus R0.

49. The method of claim 48, wherein the virus is adenovirus.

50. The method of claim 39, wherein the analyte is a pharmaceutical, a hormone, a toxin, or a heavy metal.

51. The method of claim 39, wherein the analyte is a macromolecule.

52. The method of claim 39, wherein the sample is an environmental sample.

53. The method of claim 39, wherein the sample is wastewater, tapwater, or drinking water.

54. The method of claim 39, wherein the sample is a bodily fluid from a subject.

55. A method of quantifying an analyte, the method comprising the steps of:
(a) providing the sensor of any of claims 1-31 or the system of any of claims 32-38 and a sample suspected of containing the analyte, wherein the recognition agent of the sensor is an antibody that specifically binds to the analyte;
(b) optionally conditioning the sample by filtration, dilution, concentration, dialysis, centrifugation, or another method;
(c) contacting the sample, or the conditioned sample, with the SWNT of the sensor and allowing the analyte to bind to the antibody; and
(d) determining a change in conductance or resistance of the SWNT in the sensor.

56. The method of claim 55, wherein step (d) comprises applying a series of different step voltages and measuring the current at each voltage.

57. The method of claim 56, wherein the voltages are in the range 0 to about 100 mV.

58. The method of claim 55, further comprising calibrating the sensor using a series of standard solutions having known concentrations of the analyte.

59. The method of claim 55 capable of quantifying the analyte in less than 30 min.

60. The method of claim 55, wherein the analyte is a microbe.

61. The method of claim 60, wherein the microbe is a virus, bacterium, fungus, or protist.

62. The method of claim 55, wherein the analyte is a bacterium, and the method provides a linear response over the range from about 1 to about 1,000,000 CFU/mL using a plot of log(bacteria concentration) vs. ΔR/R0, where R0 is the SWNT resistance prior to adding the sample, and ΔR is the SWNT resistance in the presence of the sample minus R0.

63. The method of claim 62, wherein the bacterium is Escherichia coli.

64. The method of claim 60, wherein the microbe is virus, and the method provide a linear response over the range from about 10 to about 10,000 PFU/mL using a plot of log(virus concentration) vs. ΔR/R0, where R0 is the SWNT resistance prior to adding the sample, and ΔR is the SWNT resistance in the presence of the sample minus R0.

65. The method of claim 64, wherein the virus is adenovirus.

66. The method of claim 55, wherein the analyte is a pharmaceutical, a hormone, a toxin, or a heavy metal.

67. The method of claim 55, wherein the analyte is a macromolecule.

68. The method of claim 55, wherein the sample is an environmental sample.

69. The method of claim 55, wherein the sample is wastewater, tapwater, or drinking water.

70. The method of claim 55, wherein the sample is a bodily fluid from a subject.

71. A method of fabricating the sensor for quantifying an analyte, the method comprising the steps of:
(a) depositing a pair of electrodes on an insulating surface of a substrate, with a gap between the electrodes;
(b) depositing one or more SWNT to form a conductive bridge between the electrodes and across the gap;
(c) functionalizing the SWNT non-covalently with a recognition agent capable of specifically recognizing said analyte;
wherein a conductometric circuit connected to said electrodes detects changes in resistance of the SWNT in relation to an amount of analyte present in the sample.

72. The method of claim 71, wherein in step (b) one or more SWNT are deposited using an electric field-assisted directed assembly process.

73. The method of claim 71, wherein the recognition agent is covalently attached to a coupling agent that is non-covalently attached to the SWNT via π-π stacking interactions.

74. The method of claim 73, wherein the coupling agent is 1-pyrenebutanoic acid succinimidyl ester.

75. The method of claim 71, further comprising fabricating a conductometric circuit on the substrate.
